(19) United States
(12) Patent Application Publication
(10) Pub. No.: US 2021/0378500 A1
(43) Pub. Date: Dec. 9, 2021

(54) STOMACH TUBE

(71) Applicant: Naser DIB, Warszawa (PL)

(72) Inventor: Naser DIB, Warszawa (PL)

(21) Appl. No.: 17/286,897

(22) PCT Filed: Oct. 22, 2019
(86) PCT No.: PCT/IB19/59008

§ 371 Date: Apr. 20, 2021

(30) Foreign Application Priority Data
Nov. 11, 2018 (PL) PL427731

(51) Int. Cl. A61B 1/273; A61B 1/018; A61B 1/00

(52) U.S. Cl. CPC A61B 1/2736; A61B 1/018; A61B 1/00034; A61B 1/00126; A61B 1/00165

(57) ABSTRACT

The stomach tube is a transparent conduit in the form of a plastic tube which is equipped with an optical fibre with illuminating external surface. Its internal end is placed in the section of the stomach tube for insertion in the patient's stomach. An external end for connection with a light source is provided outside the stomach tube in the section of the stomach tube remaining outside the patient's body. The conduit of the stomach tube in its interior includes a first channel which is a transport channel, and a second channel in which the optical fibre is located. The stomach tube is intended for use in bariatric surgery procedures, and in particular in the sleeve gastrectomy procedure.

### CROSS-REFERENCE TO RELATED APPLICATIONS

See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The subject matter of the invention is a stomach tube for medical use in bariatric surgery procedures, particularly in sleeve gastrectomy.

### 2. Description of Related Art Including Information Disclosed Under 37 CFR 1.97 and 37 CFR 1.98

Prior art stomach tube is in the form of an elastic or rigid tube inserted in the patient's stomach or intestines through the nose or abdomen, particularly to enable his feeding. Enteral nutrition is a form of nutritional therapy which comprises delivering nutrients, water, protein, electrolytes, and trace elements to the lumen of the gastrointestinal (GI) tract through routes other than oral administration. Such tube is useful in patients with exacerbated gastro-oesophageal reflux, gastroparesis, intensive care unit patients (as it allows administration of medicines), with severe trauma, at risk of chronic intestinal paralysis, and with severe and acute pancreatitis. Tubes of this type are available in various sizes.

The prior art also describes a wide tube useful in gastric lavage to remove poisons or toxic substances from the stomach. Gastric lavage is effective only when less than an hour elapsed since strong toxin ingestion. In case of medicines, especially of prolonged release, gastric lavage is effective within 4 hours of ingestion; and in case of ingestion of Amanita phalloides (commonly known as the death cap mushroom) the procedure is effective if administered within 10 or more hours. The tube is inserted into the stomach after having been lubricated with lidocaine, and then with a syringe or funnel aqueous solution of 0.45% NaCl is fed which flows through the stomach tube into the stomach. After a while, the tube end is lowered below the patient's body level, which causes an outflow of gastric contents. The procedure is repeated until clear gastric washings are obtained.

The prior art also describes a stomach tube for surgical procedures, in particular for laparoscopic sleeve gastrectomy. Such operation involves resection of ⅗ of the stomach. The tube is in the form of an elastic conduit which is inserted through the mouth into the prepyloric segment of the patient's stomach and then by manipulating the instruments is positioned along the lesser curvature of the stomach. During the procedure, the left part of the stomach is cut off, with an incision made along the tube.

The tube used traditionally in such procedures had no illumination. Due to the limited visibility in the course of the procedure there is a risk of tube damage while stomach cutting off with an endostapler. Associated complications can be life-threatening for the patient. When the resection is completed, methyl blue is fed via the tube in order to test for stomach wall leaking. After the test, the dye is extracted through the tube.

The utility model application W.126183 describes a LED-illuminated stomach tube useful in particular in laparoscopic sleeve gastrectomy procedures. This tube, similarly to traditionally used tubes, has the form of an elastic tube, but has LED illumination. LEDs are placed in the wall of the stomach tube. Illumination may be fitted along the entire length of the tube or in the distal segment only. LED illumination helps locate the tube in the patient's abdomen, as the light projects through the patient's stomach wall. This tube is also useful for gastric decompression and leak testing after surgical procedures.

Tubes made out of plastics, such as PVC, silicone or polyurethane, are well known in the art.

Prior art non-illuminated stomach tubes do not protect the stomach tube from damage during the surgery. Illuminated stomach tubes according to prior art are difficult to implement in terms of embedding LEDs within the tube wall, as well as in terms of arrangement of diodes power wires.

The object of the invention is to avoid the disadvantages of known stomach tubes as well as to provide a new type of stomach tube that is safe for bariatric surgery use.

### BRIEF SUMMARY OF THE INVENTION

The stomach tube for bariatric surgery according to the invention is in the form of conduit made as transparent plastic tube, wherein the stomach tube placed inside the conduit is equipped with optical fibre with illuminating external surface, having its internal end placed in the section of the stomach tube for insertion into the patient's stomach, and an external end for connecting with a light source provided outside the stomach tube in the tube section remaining outside the patient's body. The conduit of the stomach tube in its interior comprises a first channel which is a transport channel, and a second channel in which the optical fibre is located.

Preferably, the optical fibre has the illuminating surface along its entire length.

Alternatively, the illuminating surface of the optical fibre is preferably present only in a distal part at the internal end.

Preferably also the optical fibre has the illuminating surface only in the distal part at the internal end and the external surface of the optical fibre has notches in this section.

Preferably, the light source is connected with the mains adapter.

Preferably also the light source can be powered from a rechargeable battery.

### BRIEF DESCRIPTION OF THE SEVERAL VIEWS OF THE DRAWINGS

The subject of the present invention is now illustrated by reference to the accompanying drawing, of which

FIG. 1 presents magnified proximal and distal parts of the stomach tube;

FIG. 2 presents the stomach tube inserted in the patient's body;

FIG. 3 presents the distal part of the stomach tube inserted in the patient's stomach; and

FIG. 4 is a cross-sectional view of the stomach tube along AA line shown on FIG. 1.

### DETAILED DESCRIPTION OF THE INVENTION

The stomach tube 1 according to the invention is intended to be used in bariatric surgery procedures, and in particular in the sleeve gastrectomy as an obesity treatment procedure.

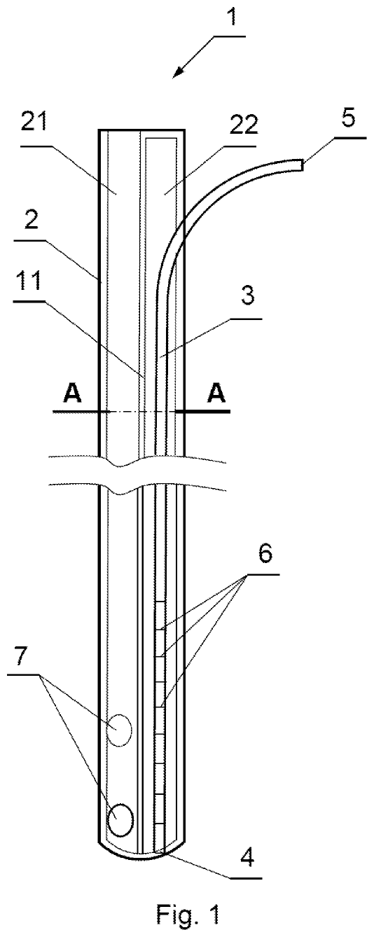

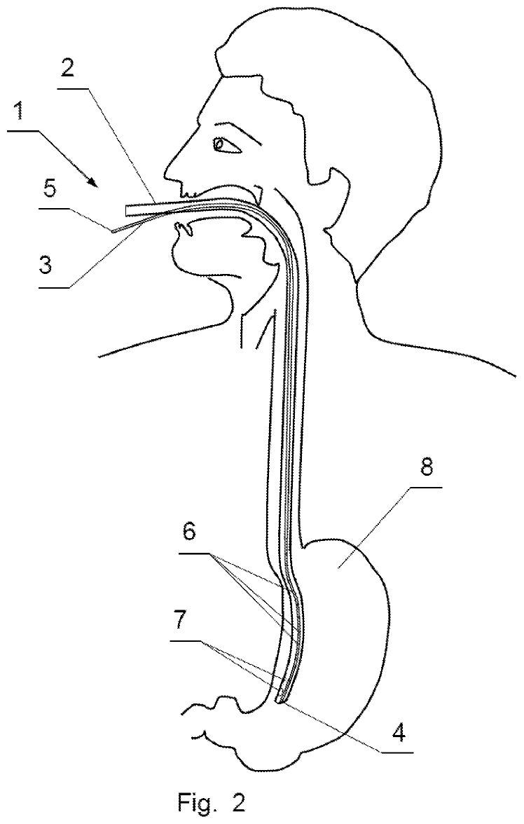

In the embodiment displayed in FIGS. 1, 2, and 3, a stomach tube 1 is in the form of transparent conduit 2 in the form of an elastic tube made out of plastic, and is provided with an optical fibre 3 which emits light through its external surface. An internal end 4 of the optical fibre 3 is placed in the section of the stomach tube 1 to be inserted into a patient's stomach 8, and an external end 5 of the optical fibre 3, intended for connecting with a light source, is provided outside the stomach tube 1 in the stomach tube 1 section remaining outside the patient's body. The conduit 2 of the stomach tube 1 in its interior comprises the first channel 21, which is a transport channel, and the second channel 22, in which the optical fibre 3 is placed.

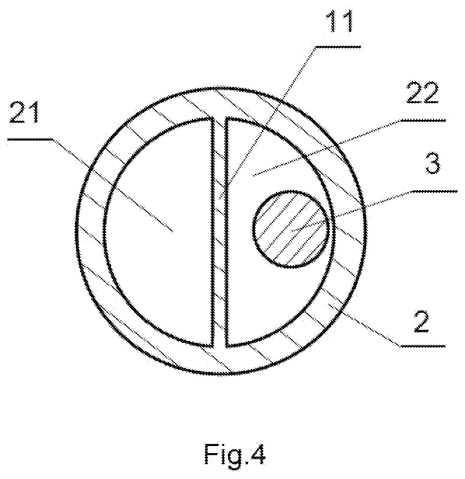

The conduit 2 illustrated in FIG. 4 has a circular cross-section, and the first channel 21 is separated from the second channel 22 by an internal wall 11 constructed from the same material as the external wall of the conduit 2. In this embodiment, the cross-section of the first channel 21 is equal to the cross-section of the second channel 22.

In the embodiment shown in FIGS. 1, 2, and 3, the optical fibre 3 has the illuminating surface along its entire length. Moreover, at the end section of the optical fibre 3 at the internal end 4, the external surface of the optical fibre 3 in this section has notches 6 enhancing emission of light from the lateral surface of the optical fibre 3.

It is apparent that in other embodiments the stomach tube 1 may be provided with the optical fibre 3 with an illuminating surface only in the distal section at the internal end 4 of the optical fibre 3.

In order for the optical fibre 3 of the fibre stomach tube 1 to emit light, it is necessary to connect the optical fibre 3 to a light source. This is achieved through the external end 5 of the optical fibre 3. The light source is connected with the mains adapter. Alternatively, the light source may be powered from a rechargeable or non-rechargeable battery.

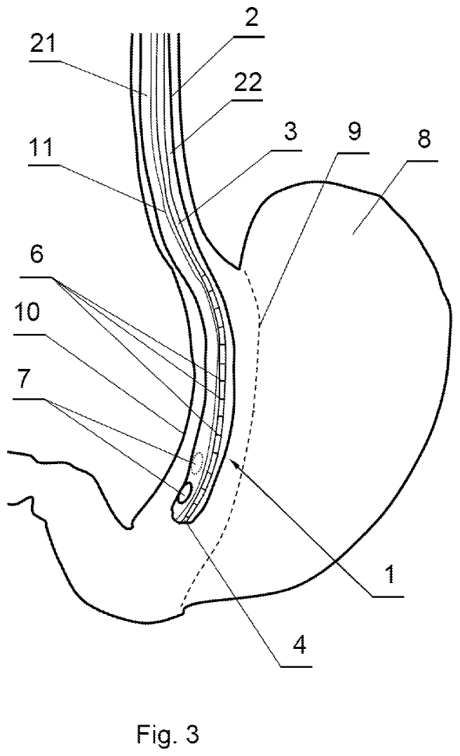

In the embodiment shown in FIGS. 1 and 3, the second channel 22 of the stomach tube 1 is closed from the top, and the optical fibre 3 is introduced to the second channel 22 via an opening in the external wall of the conduit 2 in the upper section of the conduit 2. The external end 5 of the optical fibre 3 remains outside the conduit 2, and its internal end 4 extends to the distal, lower end of the second channel 22. The first channel 21 and the second channel 22 at the distal end are closed (capped) in a manner well known in the art. The capping of the first channel 21 and the second channel 22 at the distal end renders the internal end 4 of the optical fibre 3 immobile. The first channel 21 is opened from the top. In the distal section of the stomach tube 1, the external wall of the conduit 2 of the first channel 21, being the transport channel, is provided with two circular transport openings 7. The first channel 21 is a pass-through channel and may be used to deliver liquids to the stomach 8 and out of the stomach 8, while the second channel 22, into which the optical fibre 3 is introduced through the side wall of the conduit 2, is closed from the top and from the bottom and separated from the first channel 21 by the internal wall 11.

During the laparoscopic sleeve gastrectomy procedure the stomach tube 1 is introduced through the mouth and oesophagus to the prepyloric segment of the patient's stomach 8. During the procedure, the stomach tube 1 is manipulated with surgical instruments to align it parallelly to the lesser curvature 10 of the stomach 8. Illumination of the stomach tube 1 during the procedure facilitates finding its location inside the stomach 8, which in turn reduces the risk of stomach tube 1 damage from, for example, cutting it with an endostapler, which may lead to complications in the patient. Following the clipping of the stomach vessels, approximately 85% of the stomach 8 is resected, and the cutting is made along line 9 running along and in the proximity of the stomach tube 1, as illustrated in FIG. 3. The cutting is made using an endostapler, i.e., a surgical instrument which joins the edges of the remaining part of the patient's stomach 1 using staples. When the stomach 8 resection is completed, methyl blue is delivered to the remaining part of the patient's stomach 8 through the first channel 21 of the stomach tube 1 in order to test for leaking of stomach 8 walls. When the test is completed, the dye is extracted via the transport openings 7 and sucked through the first channel 21 from the patient's stomach 8.

1. A stomach tube for use in bariatric surgery, comprising:
a conduit formed as a transparent plastic tube, an optical fibre with illuminating external surface inside the conduit, with its internal end placed in the section of the stomach tube for insertion in the patient's stomach, and an external end for connection with a light source provided outside the stomach tube in the section of the stomach tube remaining outside the patient's body,
wherein the conduit of the stomach tube in its interior comprises a first channel which is a transport channel, and a second channel in which is located the optical fibre having notches in its illuminating surface.

2. The stomach tube according to claim 1, wherein the optical fibre comprises the illuminating surface along its entire length.

3. The stomach tube according to claim 1, wherein the illuminating surface of the optical fibre is present only in the distal part at the internal end.

4. The stomach tube according to claim 1, wherein the light source is connected with the main adapter.

5. The stomach tube according to claim 1, wherein the light source is powered from a rechargeable battery.
